SEQUENCE LISTING (1 sequence)

SEQ ID NO: 1 - protein, 275 aa, Artificial Sequence
  Synthetic Construct
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

(19) United States
(12) Patent Application Publication
(10) Pub. No.: US 2022/0143187 A1
(43) Pub. Date: May 12, 2022

(54) PRESERVED FORMULATIONS

(71) Applicant: Eli Lilly and Company, Indianapolis, IN (US)

(72) Inventors: Dinesh Shyamdeo Mishra, Carmel, IN (US); Ken Kangyi Qian, Carmel, IN (US)

(21) Appl. No.: 17/438,692

(22) PCT Filed: Mar. 11, 2020
(86) PCT No.: PCT/US2020/022111

§ 371 Date: Sep. 13, 2021

Related U.S. Application Data
(60) Provisional application No. 62/819,096, filed on Mar. 15, 2019.

(51) Int. Cl. A61K 47/10; A61K 9/00; A61K 38/26; A61K 9/08; A61K 47/26

(52) U.S. Cl. CPC A61K 47/10; A61K 9/0019; A61K 47/26; A61K 9/08; A61K 38/26

(57) ABSTRACT

The present invention relates to preserved, surfactant-containing pharmaceutical compositions that are suitable for parenteral administration.

The present invention relates to preserved, surfactant-containing pharmaceutical compositions that are suitable for parenteral administration. The compositions include one or more preservatives, such as metacresol or phenol, one or more surfactants, such as polysorbate 80 (PS80), one or more active pharmaceutical ingredients (APIs), such as dulaglutide, and one or more solvent modifiers, such as propylene glycol (PPG), N-methyl-2-pyrrolidone (NMP), polyethylene glycol (PEG) 400 or glycerol.

Protein and peptide-based drug products typically must be administered parenterally, due to susceptibility of proteins and peptides to proteolysis in the digestive tract if administered orally, and in some cases must be formulated with nonionic surfactants, to ensure the stability of the proteins during storage and throughout in-use conditions. A limitation of such surfactant-containing formulations which require surfactant concentrations above certain levels, however, is that they cannot be sufficiently preserved for multi-use presentations, because interactions between surfactants and preservatives results in the formation of unacceptable visible precipitates. This incompatibility of surfactants and preservatives has been recognized previously. See, e.g., S. Kazmi and A. Mitchell, Interaction of Preservatives with Cetomacrogol, 23 J. PHARM. PHARMAC. 482-489 (1970); J. Blanchard, Effect of Sorbitol on Interaction of Phenolic Preservatives with Polysorbate 80, 66 J. PHARM. SCI. 10, 1471-1472 (1977); J. Blanchard, Effect of Polyols on Interaction of Paraben Preservatives with Polysorbate 80, 69 J. PHARM. SCI. 2, 169-173 (1980); R. Torosantucci, Protein-Excipient Interactions Evaluated via Nuclear Magnetic Resonance Studies in Polysorbate-Based Multidose Protein Formulations: Influence on Antimicrobial Efficacy and Potential Study Approach, 107 J. PHARM. SCI. 10, 2531-2537 (2018). A solution to that incompatibility, however, has not been described.

Therefore, currently available protein and peptide-based drug products requiring certain concentrations of surfactants as stabilizing agents are sold in non-preserved, single-use formulations. For example, dulaglutide is a glucagon-like peptide 1 (GLP-1) receptor agonist fusion protein sold under the tradename TRULICITY™ in a formulation which requires 0.20 mg/mL polysorbate 80 for stabilization purposes, but which does not include a phenolic preservative due to phase separation that would occur if a phenolic preservative were added in a concentration sufficient to meet regulatory requirements. See Highlights of Prescribing Information, TRULICITY (dulaglutide) injection, for subcutaneous use (Initial U.S. FDA Approval: 2014). Dulaglutide is therefore currently sold in a device that must be discarded after a single use, which—in comparison with preserved, multi-use products—is associated with disadvantages including increased cost of products sold (COPS) and increased physical waste.

Formulations of protein or peptide-based drug products containing surfactants in concentrations similar to that used in the current commercial formulation of dulaglutide, or preservatives in concentrations sufficient to meet regulatory requirements for sterility, but not both, have been described previously. For example, U.S. Patent Application No. 2009/0232807 describes formulations of GLP-1-Fc fusion proteins, and lists various categories and examples of excipients, including what the application describes as “solubilizers,” such as Tween 80® (also known as polysorbate 80), and preservatives, such as m-cresol. The application does not, however, provide any examples or embodiments of a formulation containing both a recited “solubilizer” and a recited preservative. U.S. Patent Application No. 20100196405 describes formulations of dulaglutide, including formulations that include polysorbate 80 in a concentration of about 0.2% (w/v). The application does not, however, describe formulations containing preservatives.

There remains a need for formulations which contain surfactants in concentrations sufficient to stabilize proteins or peptides and preservatives in concentrations sufficient to meet antimicrobial requirements for multi-use injectable products.

In one aspect, the present invention provides a composition comprising:
a) a protein or peptide;
b) A non-ionic surfactant;
c) a phenolic preservative; and
d) a solvent modifier;
   wherein the non-ionic surfactant and the phenolic preservative are present in concentrations above their concentration threshold in the absence of a solvent modifier; and wherein the solvent modifier is present in a concentration sufficient to ensure the solution remains clear.

In another aspect, the present invention provides a method for preparing a clear formulation containing a non-ionic surfactant and a phenolic preservative in concentrations above their concentration threshold in the absence of a solvent modifier, comprising including in the composition a solvent modifier.

In another aspect, the present invention provides an article of manufacture comprising an aqueous composition comprising:
a) a protein or peptide;
b) a non-ionic surfactant;
c) a phenolic preservative; and
d) a solvent modifier;
   wherein the non-ionic surfactant and the phenolic preservative are present in concentrations above their concentration threshold in the absence of a solvent modifier; and wherein the solvent modifier is present in a concentration sufficient to ensure the solution remains clear.

In another aspect, the present invention provides a method of preparing a composition comprising a non-ionic surfactant and a phenolic preservative above their concentration threshold, comprising including in the composition a solvent modifier in a concentration sufficient to ensure the composition remains clear.

As noted above, surfactants are included in the formulations of many protein or peptide-based drug products in order to stabilize the protein or peptide APIs. When used herein, the term “protein or peptide-based drug product” refers to a pharmaceutically acceptable composition for use in treating or preventing a disease or condition in a subject wherein the composition contains at least one API which is a peptide or a protein. Although peptides and proteins are sometimes distinguished by size, with peptides having between 2 and 50 amino acids and proteins having greater than 50 amino acids, the difference between the two is not relevant for the purposes of the present invention, as the formulations described herein are equally applicable to drug products containing one or more API which is a peptide or a protein. The formulations of the present invention may be applicable to a wide variety of protein or peptide-based drugs that require non-ionic surfactants for stability purposes.

A preferred drug for use in formulations of the present invention is dulaglutide, which is a human GLP-1R agonist which comprises a dimer of a GLP-1 analog fused at its C-terminus via a peptide linker to the N-terminus of an analog of an Fc portion of an immunoglobulin, and is identified by CAS registry number 923950-08-7, which provides the following chemical name: 7-37-Glucagon-like peptide I [8-glycine,22-glutamic acid,36-glycine] (synthetic human) fusion protein with peptide (synthetic 16-amino acid linker) fusion protein with immunoglobulin G4 (synthetic human Fc fragment), dimer. Each monomer of dulaglutide has the amino acid sequence set forth in SEQ ID NO:1:

```
(SEQ ID NO: 1)
                  10 20 30 40 50 60
HGEGTFTSDVSSYLEEQAAKEFIAWLVKGGGGGGGSGGGGSGGGGSAESKYGPPCPPCPA
                  70 80 90 100 110 120
PEAAGGPSVFLFPPKPKDTLMISRTPEVTCVVVDVSQEDPEVQFNWYVDGVEVHNAKTKP
                  130 140 150 160 170 180
REEQFNSTYRVVSVLTVLHQDWLNGKEYKCKVSNKGLPSSIEKTISKAKGQPREPQVYTL
                  190 200 210 220 230 240
PPSQEEMTKNQVSLTCLVKGFYPSDIAVEWESNGQPENNYKTTPPVLDSDGSFFLYSRLT
                  250 260 270
VDKSRWQEGNVFSCSVMHEALHNHYTQKSLSLSLG.
```

The two monomers are attached by disulfide bonds between the cysteine residues at positions 55 and 58 to form the dimer. Dulaglutide's structure, function, production and use in treating T2DM is described in more detail in U.S. Pat. No. 7,452,966 and U.S. Patent Application Publication No. US20100196405. When used herein, the term “dulaglutide” refers to any GLP-1R agonist protein dimer of two monomers having the amino acid sequence of SEQ ID NO:1, including any protein that is the subject of a regulatory submission seeking approval of a GLP-1R agonist product which relies in whole or part upon data submitted to a regulatory agency by Eli Lilly and Company relating to dulaglutide, regardless of whether the party seeking approval of said protein actually identifies the protein as dulaglutide or uses some other term.

Other examples of proteins or peptides which may be used in formations of the present invention include, but are not limited to, those described in the examples below, as well as other Fc fusion proteins, other GLP-1 agonists, gastric inhibitory peptide (GIP) receptor agonists, glucagon receptor agonists, peptide YY (PYY) and variants thereof, growth differentiation (GDF) factors such as GDF15 and variants thereof, amylin receptor agonists, calcitonin receptor agonists and interleukins and variants thereof.

Many proteins and peptides are susceptible to denaturation and/or aggregation when formulated in aqueous solutions, and surfactants are commonly added to formulations of such proteins and peptides to attenuate such issues. Surfactants are composed of molecules which have hydrophilic and hydrophobic portions and which tend to aggregate in aqueous solutions to form agglomerations known as micelles. Inclusion of surfactants in aqueous solutions of peptide- or protein-based pharmaceuticals decrease the surface tension of the solution and help protect the peptides or proteins from coming into contact with any oxygen in the container. Examples of surfactants disclosed for use in parenteral pharmaceutical compositions include polysorbates, such as polysorbate 20 (TWEEN® 20) and polysorbate 80 (TWEEN® 80) and block copolymers such as poloxamer 188 (CAS Number 9003-11-6, sold under trade name PLURONIC® F-68) and poloxamer 407 (PLURONIC® F127).

The formulations of the present invention include one or more non-ionic surfactants. In certain embodiments, the non-ionic surfactant is a polysorbate-type surfactant. Polysorbates are fatty acid esterified ethyoxylated sorbitans, and particular polysorbates are identified by the type of fatty acid ester associated with the polyoxyethylene sorbitan. For example, polysorbate 20 comprises a monolaurate, polysorbate 40 comprises a monopalmitate, polysorbate 60 comprises a monostearate and polysorbate 80 comprises a monooleate. Polysorbate 20 and polysorbate 80 are commonly used surfactants in pharmaceutical products for parenteral administration, and are included as the surfactant(s) in certain preferred embodiments of the present invention. In other embodiments, the non-ionic surfactant is a poloxamer. Poloxamers are block copolymers comprised of a polyxoypropylene chain and two polyoxyethylene chains, and are commonly categorized by a number indicating the mass of the polyoxypropylene core and the percent of polyoxyethylene. Examples include poloxamer 188 and poloxamer 407. Poloxamer 188, in particular, is a commonly used surfactant in pharmaceutical products for parenteral administration, and is included as the surfactant(s) in certain preferred embodiments of the present invention.

In certain preferred embodiments, the non-ionic surfactant is selected from the group consisting of polysorbate 80, polysorbate 20 and poloxamer 188. In certain embodiments, the non-ionic surfactant is polysorbate 80. In certain embodiments, the concentration of polysorbate 80 is from about 0.01 mg/mL to about 1 mg/mL. In certain embodiments, the concentration of polysorbate 80 is from about 0.05 mg/mL to about 0.5 mg/mL. In certain embodiments, the concentration of polysorbate 80 is from about 0.1 mg/mL to about 0.4 mg/mL. In certain preferred embodiments, the concentration of polysorbate 80 is from about 0.2 mg/mL to about 3 mg/mL. In certain embodiments, the concentration of polysorbate 80 is selected from the group consisting of about 0.2 mg/mL and about 0.25 mg/mL. In certain embodiments, the concentration of polysorbate 80 is about 0.2 mg/mL. In certain embodiments, the concentration of polysorbate 80 is about 0.25 mg/mL. In certain embodiments, the non-ionic surfactant is polysorbate 20. In certain embodiments, the concentration of polysorbate 20 is from about 0.01 mg/mL to about 1 mg/mL. In certain embodiments, the concentration of polysorbate 20 is from about 0.05 mg/mL to about 0.5 mg/mL. In certain embodiments, the concentration of polysorbate 20 is from about 0.1 mg/mL to about 0.4 mg/mL. In certain embodiments, the non-ionic surfactant is poloxamer 188. In certain embodiments, the concentration of poloxamer 188 ranges from about 0.01 to about 2 mg/mL. In certain embodiments, the concentration of poloxamer 188 ranges from about 0.01 to about 2 mg/mL. In certain embodiments, the concentration of poloxamer 188 ranges from about 0.5 to about 1.5 mg/mL. These embodiments should not be construed as limiting, however, as persons skilled in the art are capable of identifying the identity and concentration of surfactant needed to provide sufficient stabilizing effects in a given composition.

The formulations of the present invention also include one or more preservatives, which are added to provide anti-microbial properties. The compositions are sterile when first produced, however, when the composition is provided in a multi-use vial or cartridge, an anti-microbial preservative compound or mixture of compounds that is compatible with the other components of the formulation is typically added at sufficient strength to meet regulatory and pharmacopoeial anti-microbial preservative requirements, such as those published by the European Pharmacopeia (E.P.) and the United States Pharmacopeia (USP). See European Pharmacopoeia, edition 9, section 5.1.3, Efficacy of Antimicrobial Preservation; United States Pharmacopeia. USP <51>, Antimicrobial effectiveness testing, Rockville, Md.

Commonly used preservatives in pharmaceutical products suitable for multiple-use parenteral administration include phenolic compounds, or mixtures of such compounds. Specific examples include phenol (CAS No. 108-95-2, molecular formula C₆H₅OH, molecular weight 94.11), m-cresol (CAS No. 108-39-4, molecular formula C₇H₈O, molecular weight 108.14), benzyl alcohol (CAS #: 100-51-6, molecular formula C₇H₈O, molecular weight 108.14 g/mol) and phenoxyethanol (CAS #: 122-99-6, molecular formula C₈H₁₀O₂, molecular weight 138.17 g/mol). In certain embodiments of the present invention, the phenolic preservative is selected from the group consisting of phenol and m-cresol and mixtures thereof. The concentration of preservative needed to meet regulatory requirements for multi-use products depends on multiple factors, including but not limited to the identity of the phenolic preservative used and the pH of the solution. In certain embodiments, the phenolic preservative is phenoxyethanol, which is present in a concentration of about 10 to about 15 mg/mL. In certain embodiments, the phenolic preservative is benzyl alcohol. In certain embodiments, the phenolic preservative is benzyl alcohol, which is present in a concentration of about 10 mg/mL. In certain embodiments, the phenolic preservative is phenol. In certain embodiments, the phenolic preservative is phenol, which is present in a concentration of about 1 to about 10 mg/mL. In certain embodiments, the phenolic preservative is phenol, which is present in a concentration of about 3 to about 6 mg/mL. In certain embodiments, the phenolic preservative is phenol in a concentration of at least about 3 mg/mL. In certain embodiments, the phenolic preservative is phenol in a concentration selected from the group consisting of 3, 3.5, 4, 4.5 or 5 mg/mL. In a preferred embodiment, the phenolic preservative is phenol in a concentration of about 4 mg/mL. In certain embodiments, the phenolic preservative is m-cresol. In certain embodiments, the phenolic preservative is m-cresol, which is present in a concentration of about 0.1 to about 10 mg/mL. In certain embodiments, the phenolic preservative is m-cresol, which is present in a concentration of about 2 to about 6 mg/mL. In certain embodiments, the phenolic preservative is m-cresol, which is present in a concentration of about 3.5 to about 5.5 mg/mL. In certain embodiments, the phenolic preservative is m-cresol, which is present in a concentration of about 3.15 mg/mL. In other embodiments, the phenolic preservative is a mixture of phenol and m-cresol. In certain embodiments, the phenolic preservative is a mixture of phenol and m-cresol wherein the phenol is present in a concentration of about 1 to about 5 mg/mL and the m-cresol is present in a concentration of about 0.1 to about 3.5 mg/mL. In certain embodiments, the phenolic preservative is a mixture of phenol and m-cresol wherein the phenol is present in a concentration of about 1.5 and the m-cresol is present in a concentration of 1.58 mg/mL. In certain embodiments, the phenolic preservative is a mixture of phenol and m-cresol wherein the phenol is present in a concentration of about 2 and the m-cresol is present in a concentration of about 1.58 mg/mL. In certain embodiments, the phenolic preservative is a mixture of phenol and m-cresol wherein the phenol is present in a concentration of about 3.5 and the m-cresol is present in a concentration of about 0.32 mg/mL. In certain embodiments, the phenolic preservative is a mixture of phenol and m-cresol wherein the phenol is present in a concentration of about 3.5 mg/mL and the m-cresol is present in a concentration of about 0.63 mg/mL. These embodiments should not be construed as limiting, however, as persons skilled in the art are capable of selecting a phenolic preservative and concentration thereof needed to meet regulatory requirements using known techniques. See, e.g., European Pharmacopoeia, edition 9, section 5.01.03 “Efficacy of Antimicrobial Preservation;” US Pharmacopoeia, USP 40-NF 35, Chapter <51>“Antimicrobial Effectiveness Testing;” see, e.g., Meyer, B. K., et al., Antimicrobial Preservative use in Parenteral Products: Past and Present, J. PHARM. SCI., Vol. 96, No. 12 (2007).

When surfactants and preservatives are both included in a composition in certain concentrations, however, they interact in such a way that results in a phase separation, resulting in the formation of unacceptable visible cloudiness or turbidity. Without wishing to be bound by theory, it is believed that this phenomenon occurs when molecules of the phenolic preservative associate with micelles of the non-ionic surfactant through bridging attraction. See, e.g., Chen, J., et al., From the depletion attraction to the bridging attraction: The effect of solvent molecules on the effective colloidal interactions, THE JOURNAL OF CHEMICAL PHYSICS 2015, 142, 084904; Jie, C., et al., Size effects of solvent molecules on the phase behavior and effective interaction of colloidal systems with the bridging attraction. JOURNAL OF PHYSICS: CONDENSED MATTER 2016, 28, (45), 455102; Yuan, G.; Luo, J.; Han, C. C.; Liu, Y. Gelation transitions of colloidal systems with bridging attractions. PHYSICAL REVIEW E 2016, 94, (4), 040601. This causes multiple surfactant micelles to become associated and consequently precipitate out of solution. Skilled persons will understand that micelles are assemblies of surfactant molecules wherein the hydrophilic portions of the non-ionic surfactant molecules form an outer surface or shell surrounding the hydrophobic portions, which are protected from the aqueous solvent by the outer surface, or shell formed by the hydrophilic portions. The concentration of surfactant at which such micelles are formed is known as the critical micelle concentration, or CMC, and may be determined using techniques known in the art. See, e.g., Kerwin, B. A. Polysorbates 20 and 80 used in the formulation of protein biotherapeutics: Structure and degradation pathways. JOURNAL OF PHARMACEUTICAL SCIENCES 2008, 97, (8), 2924-2935. Again without wishing to be bound by theory, it is believed that the use of a solvent modifier as described herein inhibits the bridging attraction between preservative molecules and surfactant micelles.

Regardless of the specifics of the mechanism, however, phase separation occurs when the combined concentrations of surfactants and preservatives in a given composition are at or above what is referred to herein as their “concentration threshold,” which refers to the concentration at which a combination of surfactants and preservatives, in the absence of a solvent modifier, results in phase separation leading to formation of or a cloudy or milky appearance. There is no universal concentration threshold that can be generally applied to any surfactant+preservative combination. Instead, the concentration threshold depends on specifics of the formulation in question, including in particular the identities of the surfactant(s) and preservative(s).

The concentration threshold for a given surfactant+preservative combination in any given formulation may be determined by persons of skilled in the art using known methods, including in particular visual observation, although quantitative analyses, such as the turbidity analyses described in the examples below may also be used. See, e.g., European Pharmacopoeia 7.0, Section 2.2.1, Clarity and Degree of Opalescence of Liquids. Other analyses, which may not directly reflect the formation of visible phase separation, but which may be relevant to the potential in a given composition for the ultimate development or formation of visible phase separation, include: size exclusion chromatography (SEC), analysis with a high accuracy liquid particle counter (HIAC), and micro-flow imaging (MFI).

In addition, while visually detectable phase separation in some compositions with surfactant and preservative combinations above the concentration threshold occurs essentially immediately upon combination of the surfactant and preservative, in other compositions phase separation does not become visually apparent until some time after the formulation has been prepared. For example, it has been observed that visually detectable phase separation in m-cresol-containing formulation occurs almost immediately, but in certain phenol-containing formulations does not become visually detectable until up to approximately 15 minutes after the formulation has been prepared. Thus, confirmation that a solvent modifier has sufficiently attenuated phase separation for a phenolic preservative surfactant combination otherwise above its concentration threshold, the visual appearance of the formulation must be inspected at least 10, and preferably at least 15 minutes after the formulation has been prepared.

As noted above, the concentration threshold for a given surfactant+preservative combination depends on both the identities and concentrations of surfactant(s) and preservative(s), and certain commercial products include both surfactants and preservatives yet remain clear and colorless because the surfactant+preservative combinations in those products are below their concentration thresholds. For example, the formulation of insulin glargine sold under the tradename LANTUS® includes 0.02 mg/mL polysorbate 20 and 2.7 mg/mL m-cresol and the formulation of insulin glulisine sold under the tradename APIDRA® includes 0.01 mg/mL polysorbate 20 and 3.15 mg/mL m-cresol, yet both of these formulations are clear because the combined concentrations of polysorbate 20 and m-cresol in each case are below the concentration threshold for this particular combination. Indeed, as shown in the Examples below, for formulations containing m-cresol in a concentration of 3.15 m-cresol, phase separation does not occurs when polysorbate 20 is included in concentrations at or below about 2 times its CMC but does occur at concentrations at or above about 5 times its CMC.

When used herein, the term “phase separation” refers to the formation of physical particulates that precipitate out of solution. The presence or absence of the occurrence of phase separation in a given composition may be determined visually—i.e., as indicated by a cloudy or milky, as opposed to clear, appearance—or through analytical techniques known to those skilled in the art. Similarly, when used herein, the term “clear” refers to a solution that is transparent, does not have a cloudy or milky appearance, and does not contain visibly detectable solid particles of material. The determination of whether a formulation is clear and particulate-free may be determined visually, although analytical techniques known to those skilled in the art may be used.

The present invention involves the use of solvent modifiers to attenuate the occurrence of phase separation in a composition wherein surfactant(s) and preservative(s) are included in concentrations otherwise (i.e., in the absence of a solvent modifier) at or above their concentration threshold. Compounds which may be used as solvent modifiers in formulations of the present invention include PPG (CAS No. 57-55-6, molecular formula C₃H₈O₂, molecular weight 76.095), NMP (CAS No. 872-50-4, molecular formula C₅H₉NO, molecular weight 99.133) and PEG 400 (CAS No. 25322-68-3, molecular formula C₂ₙH₄ₙ₊₂Oₙ₊₁, n=8.2 to 9.1, molecular weight 380-420 g/mol) glycerol (CAS No. 56-81-5, molecular formula C₃H₈O₃, molecular weight 92.09382).

It should be noted that the compounds identified in the preceding paragraph, which may be used as solvent modifiers in formulations of the present invention, are in some cases commonly used excipients in pharmaceutical formulations, and may have functions other than use as solvent modifiers in formulations of the present invention. For example, glycerol is a commonly used agent used for isotonicity purposes, and is included in formulations of insulin-containing products such as LANTUS® (insulin glargine), APIDRA® (insulin glulisine), HUMALOG® (insulin lispro), NOVOLOG® (insulin aspart), TRESIBA® (insulin degludec), HUMULIN® (human insulin), and TOUJEO® (insulin glargine). Those insulin-containing products, however, either do not include any surfactants, or do include surfactants, but below their concentration threshold in combination with the phenolic preservative(s) in those formulations. Similarly, PPG is also a commonly used pharmaceutical excipient for functions other than use as a solvent modifier, e.g., VICTOZA® (liraglutide) includes 14 mg/mL PPG but does not contain a non-ionic surfactant. PEG400 is also a common excipient, and is included, for example, in ATIVAN® (lorazepam), but that product does not contain a non-ionic surfactant. Finally, although less commonly used than glycerol or PPG, NMP is used in a product called ELIGARD (leuprolide acetate), but that product is non-aqueous and does not contain a phenolic preservative or surfactant.

With respect to concentrations of solvent modifiers needed to attenuate phase separation where surfactants and preservatives are included in concentrations above their concentration threshold, just as concentration thresholds vary for given surfactant+preservative combinations, so does the concentration of solvent modifier needed depend on multiple variables, including the identities and concentrations of: (a) the particular surfactant(s) and preservative(s) used; (b) the particular solvent modifier(s) being used; and (c) other excipients in the formulation, especially tonicity agents as described in more detail below. In certain embodiments of the present invention, the solvent modifier is glycerol. In certain embodiments of the present invention, the solvent modifier is glycerol, which is present in a concentration from about 10 to about 100 mg/mL. In certain embodiments, the concentration of glycerol is about 20 to about 80 mg/mL. In certain embodiments, the concentration of glycerol is selected from the group consisting of about 20, about 25 or about 80 mg/mL. In certain embodiments, the concentration of glycerol is about 20 mg/mL. In certain embodiments of the present invention, the solvent modifier is PPG. In certain embodiments of the present invention, the solvent modifier is PPG, which is present in a concentration of about 10 to about 100 mg/mL. In certain embodiments, the concentration of PPG is from about 15 to about 60 mg/mL. In certain embodiments, the concentration of PPG is selected from the group consisting of about 15, about 20 or about 60 mg/mL. In certain embodiments, the concentration of PPG is about 15 mg/mL. In certain embodiments of the present invention, the solvent modifier is NMP. In certain embodiments of the present invention, the solvent modifier is NMP, which is present in a concentration from about 10 mg/mL to about 100 mg/mL. In certain embodiments, the concentration of NMP is from about 20 to about 90 mg/mL. In certain embodiments, the concentration of NMP is from about 27 to about 80 mg/mL. In certain embodiments, the concentration of NMP is selected from the group consisting of about 27, about 54 and about 80 mg/mL. In certain embodiments of the present invention, the solvent modifier is PEG 400. In certain embodiments of the present invention, the solvent modifier is PEG 400, which is present in a concentration from about 5 to about 150 mg/mL. In certain embodiments, the concentration of PEG 400 is from about 40 to about 120 mg/mL. In certain embodiments, the concentration of PEG 400 is selected from the group consisting of about 40, about 80, about 110 and about 120 mg/mL. These concentrations should not be construed as limiting, however, as selecting an appropriate concentration of solvent modifier to use in a given composition may be readily determined by a skilled person using known techniques, including visual observation and turbidity and particulate analyses such as those described in the examples below.

In addition to attenuating incompatibility between surfactant and preservative, solvent modifiers may have additional functions in certain compositions, including in particular as a tonicity agent. Because the formulations of the present invention are intended for parenteral administration, it is desirable to approximately match the tonicity (i.e., osmolality) of body fluids at the injection site as closely as possible when administering the compositions because solutions that are not approximately isotonic with body fluids can produce a painful stinging sensation when administered. If the osmolality of a composition is sufficiently less than the osmolality of the tissue (for blood, about 300 mOsmol/kg; the European Pharmacopeial requirement for osmolality is >240 mOsmol/kg), then the tonicity of composition should be raised to about 300 mOsmol/kg. Such an effect could be achieved through the addition of a sufficient concentration of a solvent modifier, as glycerol and PPG are examples of solvent modifiers for use in formulations of the present invention, but are also commonly used as tonicity agents in parenteral products. Thus, glycerol and/or PPG maybe used in compositions of the present invention to function both as a solvent modifier and/or as a tonicity agent. For example, in the dulaglutide-containing compositions described in the Examples below, glycerol and PPG have been added in concentrations sufficient to both raise the tonicity of the compositions to be approximately isotonic with body fluids at the sites of injection and to attenuate incompatibility between the surfactant(s) and preservative(s) in those compositions.

Raising the tonicity of a composition that is less than the osmolality of the tissue can also be accomplished by adding an additional tonicity agent. Commonly used tonicity agents, however, include sodium chloride and mannitol, and it has been discovered that, in certain formulations, these agents may exacerbate the surfactant-preservative interactions that lead to phase separation, thus lowering the minimum concentrations of surfactant and/or preservative that reach the concentration threshold and/or requiring higher concentrations of solvent modifiers to avoid phase separation. In any event, if the addition of a tonicity agent is required, the amount of tonicity agent to add is readily determined using standard techniques. Remington: The Science and Practice of Pharmacy, David B. Troy and Paul Beringer, eds., Lippincott Williams & Wilkins, 2006, pp. 257-259; Remington: Essentials of Pharmaceutics, Linda Ed Felton, Pharmaceutical Press, 2013, pp. 277-300. Moreover, if the addition of a tonicity agent such as sodium chloride or mannitol is required, and if its addition exacerbates the surfactant-preservative interaction, the amount of solvent modifier needed to be added to prevent unwanted phase separation may be readily determined by persons of skill in the art using known techniques such as those described in the examples below.

As noted above, the concentrations of surfactant, preservative and solvent modifier for use in formulations of the present invention may be determined by persons skilled in the art using known techniques such as those described in the Examples below. For example, a formulator seeking to prepare a multi-use formulation of a protein or peptide-based drug product may in some cases first determine the identity and concentration of a non-ionic surfactant needed to provide sufficient stabilizing effects, then determine the identity and concentration of preservative needed to provide sufficient antimicrobial capacity, and observe whether phase separation has occurred. If no phase separation has occurred, the non-ionic surfactant+preservative combination is below its concentration threshold and no solvent modifier is needed. If phase separation has occurred, the formulator will then either determine whether a different surfactant+preservative combination may be used or turn to determining the identity and concentration of a solvent modifier according the present invention which will prevent such phase separation from occurring for that particular combination. Alternatively, the formulator may instead first determine the identity and concentration of preservative needed to provide antimicrobial capacity, then determine the identity and concentration of surfactant needed to provide sufficient stabilizing effects, then observe whether phase occurred when those excipients are combined. As with the previous scenario, if no phase separation has occurred, the surfactant+preservative combination is below its concentration threshold and no solvent modifier is needed. However, if phase separation has occurred, and if an alternative preservative+surfactant combination that avoids such phase separation cannot be identified, the formulator will turn to determining the identity and concentration of a solvent modifier according to the present invention.

In certain embodiments, formulations of the present invention include one or more buffers to control the pH, and the identity and concentration of any buffer(s) used may in certain cases be relevant to determining the concentration threshold of a given surfactant+preservative system and/or solvent modifier needed to avoid phase separation for that system is. A “buffer” is a substance that resists changes in pH by the action of its acid-base conjugate components. In certain embodiments, formulations of the present invention have a pH from about 4 to about 8, preferably, between about 5.5 and about 7.5, more preferably between about 6.0 and 7.0. In certain preferred embodiments, formulations of the present invention have a pH of about 6.5. In certain preferred embodiments, formulations of the present invention have a pH of about 7. Buffers suitable for controlling the pH of the compositions of the present invention in the desired range include, but are not limited to agents such as phosphate, acetate, citrate, or acids thereof, arginine, TRIS, and histidine buffers, as well as combinations thereof. “TRIS” refers to 2-amino-2-hydroxymethyl-1,3,-propanediol, and to any pharmacologically acceptable salt thereof. The free base and the hydrochloride form (i.e., TRIS-HCl) are two common forms of TRIS. TRIS is also known in the art as trimethylol aminomethane, tromethamine, and tris(hydroxymethyl) aminomethane. Preferred buffers in the composition of the present invention are citrate, or citric acid, and phosphate. In view of the potential relevance of any buffer to determination of concentration threshold and/or solvent modifier, a formulator may wish to determine the buffer needed before determining the identities and concentrations of the surfactants and/or preservatives to be used as described in the preceding paragraph.

The above description pertains to how a formulator may determine the identities and concentrations of surfactant, preservative and solvent modifier to be included in a formulation, but not necessarily how the formulation will ultimately be put together once those identities and concentrations have been determined. Although the order of operations in terms of which component is added in which order may have some variations, the solvent modifier will typically be added before the full concentrations of both the phenolic preservative and surfactant have been added—i.e., before any phase separation has occurred. In certain preferred embodiments, the solvent modifier will be the first component added to the formulation, followed by the phenolic preservative, followed by the protein or peptide, followed by the surfactant.

In addition to the components described above, formulations of the present invention may contain other excipients. For example, certain protein or peptide-based drug products may require an additional stabilizing agent due to sensitivity to oxidation or trace metals. Such stabilizing agents include, respectively, antioxidants, such as methionine, or chelating agents, such as EDTA.

Proteins and peptides have low oral bioavailability due to susceptibility to proteolysis and poor absorption in the gastrointestinal tract, so most proteins and peptides are administered parenterally. The formulations of the present invention are intended for parenteral administration, which may include administration by intravenous (IV) injection, subcutaneous (SC) injection, intramuscular (IM) injection, or intraperitoneal (IP) injection. In preferred embodiments, formulations of the present invention are designed for SC injection. Because the formulations of the present invention are suitable for multi-use administration, they are typically provided in a container-closure system, such as a vial or a cartridge, from which multiple doses may be withdrawn and administered. Formulations of the present invention may, for example, be provided in a vial, from which multiple doses for administration to a patient may be withdrawn by syringe. Formulations of the present invention may also be provided in a cartridge for use in a pen device, from which multiple doses may be administered. Formulations of the present invention may also be provided in a container closure such as a cartridge for use in an autoinjector or infusion pump capable of delivering multiple doses.

Additional embodiments of the invention are described below:

An aqueous composition comprising: a protein or peptide; a non-ionic surfactant; a phenolic preservative; and a solvent modifier.

The composition of the above embodiment wherein the composition is sterile.

The composition of any of the above embodiments, wherein the non-ionic surfactant and the phenolic preservative are present in concentrations above their concentration threshold in the absence of the solvent modifier.

The composition of any of the above embodiments, wherein the solvent modifier is present in a concentration sufficient to ensure the solution remains clear.

The composition of the above embodiment wherein the solution remains clear for at least 15 minutes. The composition of the preceding embodiment wherein the solution remains clear for at least 24 hours. The composition of the preceding embodiment wherein the solution remains clear for at least one week. The composition of the preceding embodiment wherein the solution remains clear for at least one month. The composition of the preceding embodiment wherein the solution remains clear for at least six months. The composition of the preceding embodiment wherein the solution remains clear for at least 1 year.

The composition of any of the above embodiments wherein the solution remains clear throughout shelf-life.

The composition of any of the above embodiments, wherein the solvent modifier is present in a concentration sufficient to prevent phase separation due to interaction between the non-ionic surfactant and the phenolic preservative.

The composition of any of the above embodiments, wherein the protein or peptide is present in a concentration ranging from about 0.1 to about 100 mg/mL.

The composition of any of the above embodiments, wherein the protein or peptide is present in a concentration ranging from about 0.5 to about 50 mg/mL.

The composition of any of the above embodiments, wherein the protein or peptide is present in a concentration ranging from about 1 to about 10 mg/mL.

The composition of any of the above embodiments, wherein the protein or peptide is selected from the group consisting of a GLP-1 receptor agonist, an insulin, a GIP receptor agonist, a glucagon receptor agonists, a PYY, a GDF, an amylin receptor agonist, a calcitonin receptor agonist and an interleukin. The composition of the preceding embodiment, wherein the protein or peptide is an Fc fusion protein.

The composition of any of the above embodiments wherein the protein or peptide is dulaglutide. The composition of the preceding embodiment wherein the concentration of dulaglutide is from about 1.5 to about 9 mg/mL. The composition of the preceding embodiment wherein the concentration of dulaglutide is selected from the group consisting of 1.5, 3.0, 6.0 and 9.0 mg/mL.

The composition of any of the above embodiments, wherein the non-ionic surfactant is a polysorbate-type surfactant. The composition of the preceding embodiment wherein the non-ionic surfactant is selected from the group consisting of PS20, PS80, poloxamer 188 and poloxamer 407. The composition of the preceding embodiment wherein the non-ionic surfactant is either PS20 or PS80.

The composition of any of the above embodiments wherein the non-ionic surfactant is PS80. The composition of the preceding embodiment wherein the concentration of PS80 is from about 0.01 mg/mL to about 1 mg/mL. The composition of the preceding embodiment wherein the concentration of PS80 is from about 0.05 mg/mL to about 0.5 mg/mL. The composition of the preceding embodiment wherein the concentration of PS80 is from about 0.1 mg/mL to about 0.4 mg/mL. The composition of the preceding embodiment wherein the concentration of PS80 is from about 0.2 mg/mL to about 0.3 mg/mL. The composition of the preceding embodiment wherein the concentration of polysorbate 80 is either 0.2 mg/mL or 0.25 mg/mL.

The composition of any of the above embodiments wherein the non-ionic surfactant is PS20. The composition of the preceding embodiment wherein the concentration of PS20 is greater than about 2 times its CMC. The composition of the preceding embodiment wherein the concentration of polysorbate 20 is from about 0.01 mg/mL to about 1 mg/mL. The composition of the preceding embodiment wherein the concentration of PS20 is from about 0.05 mg/mL to about 0.5 mg/mL. The composition of the preceding embodiment wherein the concentration of PS20 is from about 0.1 mg/mL to about 0.4 mg/mL.

The composition of any of the above embodiments wherein the non-ionic surfactant is poloxamer 188. The composition of the preceding embodiment wherein the concentration of poloxamer 188 ranges from about 0.01 to about 2 mg/mL. The composition of the preceding embodiment wherein the concentration of poloxamer 188 ranges from about 0.5 to about 1.5 mg/mL.

The composition of any of the above embodiments wherein the phenolic preservative is present in a concentration sufficient strength to meet regulatory and pharmacopoeial anti-microbial preservative requirements.

The composition of any of the above embodiments wherein the phenolic preservative is selected from the group consisting of phenol, m-cresol, benzyl alcohol and phenoxyethanol. The composition of the preceding embodiment, wherein the phenolic preservative is benzyl alcohol. The composition of the preceding embodiment, wherein the benzyl alcohol is present in a concentration of about 10 mg/mL.

In certain embodiments, the phenolic preservative is phenoxyethanol. The composition of the preceding embodiment, wherein the phenoxyethanol is present in a concentration of about 10 to about 15 mg/mL.

The composition of any of the above embodiments, wherein the phenolic preservative is selected from the group consisting of phenol and m-cresol and mixtures thereof.

The composition of any of the above embodiments, wherein the phenolic preservative is phenol. The composition of the preceding embodiment, wherein the concentration of phenol is from about 1 to about 10 mg/mL. The composition of the preceding embodiment, wherein the concentration of phenol is from about 3 to about 6 mg/mL. The composition of the preceding embodiment, wherein the concentration of phenol is at least about 3 mg/mL. The composition of the preceding embodiment, wherein embodiments the phenolic preservative is phenol in a concentration selected from the group consisting of 3, 3.5, 4, 4.5 or 5 mg/mL. The composition of the preceding embodiment, wherein the concentration of phenol is about 5 mg/mL.

The composition of any of the above embodiments, wherein the phenolic preservative is m-cresol. The composition of any of the above embodiments, wherein the phenolic preservative is m-cresol, which is present in a concentration of about 0.1 to about 10 mg/mL. The composition of the preceding embodiment, wherein the phenolic preservative is m-cresol, which is present in a concentration of about 2 to about 6 mg/mL. The composition of the preceding embodiment, wherein the phenolic preservative is m-cresol, which is present in a concentration of about 3.5 to about 5.5 mg/mL.

The composition of any of the above embodiments, wherein the phenolic preservative is a mixture of phenol and m-cresol. The composition of the preceding embodiment, wherein the phenolic preservative is a mixture of phenol and m-cresol wherein the phenol is present in a concentration of about 1 to about 5 mg/mL and the m-cresol is present in a concentration of about 0.1 to about 3.5 mg/mL. The composition of the preceding embodiment, wherein the phenolic preservative is a mixture of phenol and m-cresol wherein the phenol is present in a concentration between about 1.5 and about 2, and the m-cresol is present in a concentration of 1.58 mg/mL.

The composition of any of the above embodiments, wherein the phenolic preservative is a mixture of phenol and m-cresol wherein the phenol is present in a concentration of about 3.5 to about 4 mg/mL and the m-cresol is present in a concentration of about 0.32 mg/mL to about 0.63 mg/mL. The composition of the preceding embodiment, wherein the concentration of phenol is about 3.5 mg/mL and the concentration of m-cresol is about 0.32 mg/mL.

The composition of any of the above embodiments, wherein the solvent modifier is selected from the group consisting of PPG, NMP, PEG 400 and glycerol.

The composition of any of the above embodiments, wherein the solvent modifier is glycerol. The composition of any of the above embodiments, wherein the solvent modifier is glycerol, which is present in a concentration from about 10 to about 100 mg/mL. The composition of the preceding embodiment, wherein the concentration of glycerol is about 20 to about 80 mg/mL. The composition of the preceding embodiment, wherein the concentration of glycerol is selected from the group consisting of about 20, about 25 or about 80 mg/mL. The composition of the preceding embodiment, wherein the concentration of glycerol is about 20 mg/mL.

The composition of any of the above embodiments, wherein the solvent modifier is PPG. The composition of any of the above embodiments, wherein the solvent modifier is PPG, which is present in a concentration of about 10 to about 100 mg/mL. The composition of the preceding embodiment wherein the concentration of PPG is from about 15 to about 60 mg/mL. The composition of the preceding embodiment, wherein the concentration of PPG is selected from the group consisting of about 15, about 20 or about 60 mg/mL. The composition of the preceding embodiment, wherein the concentration of PPG is about 15 mg/mL.

The composition of any of the above embodiments, wherein the solvent modifier is NMP. The composition of any of the above embodiments, wherein the solvent modifier is NMP, which is present in a concentration from about 10 mg/mL to about 100 mg/mL. The composition of the preceding embodiment wherein the concentration of NMP is from about 20 to about 90 mg/mL. The composition of the preceding embodiment wherein the concentration of NMP is from about 27 to about 80 mg/mL. The composition of the preceding embodiment wherein the concentration of NMP is selected from the group consisting of about 27, about 54 and about 80 mg/mL.

The composition of any of the above embodiments wherein the solvent modifier is PEG 400. The composition of any of the above embodiments wherein the solvent modifier is PEG 400, which is present in a concentration from about 5 to about 150 mg/mL. The composition of the preceding embodiment wherein the concentration of PEG 400 is from about 40 to about 120 mg/mL. The composition of the preceding embodiment wherein the concentration of PEG 400 is selected from the group consisting of about 40, about 80, about 110 and about 120 mg/mL.

The composition of any of the above embodiments wherein the composition further comprises a tonicity agent. The composition of the preceding embodiment wherein the tonicity agent is selected from the group consisting of NaCl and mannitol.

The composition of any of the above embodiments wherein the composition further comprises a buffer. The composition of the preceding embodiment wherein the buffer is selected from the group consisting of phosphate, acetate, citrate, or acids thereof, arginine, TRIS, and histidine. The composition of the preceding embodiment wherein the buffer is phosphate. The composition of the preceding embodiment wherein the concentration of phosphate is about 10 mM

The composition of any of the above embodiments wherein the composition further comprises a buffer, which is citrate. The composition of the preceding embodiment wherein the concentration of citrate is about 10 mM.

The composition of any of the above embodiments wherein the pH of the composition is from about 4 to about 8. The composition of the preceding embodiment wherein the pH of the composition is between about 5.5 and about 7.5. The composition of the preceding embodiment wherein the pH of the composition is between about 6.0 and 7.0. The composition of the preceding embodiment wherein the pH of the composition is about 6.5 or about 7.

The composition of any of the above embodiments wherein the composition further comprises an additional stabilizing agent. The composition of the preceding embodiment wherein the additional stabilizing agent is an antioxidant or a chelating agent. The composition of the preceding embodiment wherein the antioxidant is methionine and the chelating agent is EDTA.

An aqueous composition suitable for parenteral administration comprising: dulaglutide, PS80, a solvent modifier selected from the group consisting of PPG and glycerol and a phenolic preservative selected from the group consisting of phenol, m-cresol and mixtures thereof. The composition of the preceding embodiment, wherein the dulaglutide concentration is selected from the group consisting of 1.5, 3, 6 or 9 mg/mL. The composition of the preceding embodiment wherein the concentration of PS80 is either 0.2 or 0.25 mg/mL. The composition of the preceding embodiment wherein the solvent modifier is either 15 mg/mL PPG or 20 mg/mL glycerol. The composition of the preceding embodiment wherein the phenolic preservative is either 4 mg/mL phenol or a combination of 3.5 mg/mL phenol and 0.32 mg/mL m-cresol. The composition of the preceding embodiment further comprising a buffer. The composition of the preceding embodiment wherein the buffer is citrate. The composition of the preceding embodiment wherein the concentration of citrate is 10 mM. The composition of the preceding embodiment wherein the pH of the composition is about 6.5.

A container-closure system comprising any of the above-described compositions. The container-closure system of the previous embodiment wherein the container-closure system is a vial or a cartridge.

A multiple dose pen device comprising any of the above-described compositions.

A multiple dose autoinjector comprising any of the above-described compositions.

An infusion pump comprising any of the above-described compositions.

A method of preparing any of the above-described compositions comprising preparing or obtaining a buffer, then adding the solvent modifier, then adding the phenolic preservative, then adding the protein or peptide-based API, then adding the surfactant.

A method of preparing an aqueous composition suitable for parenteral administration comprising a non-ionic surfactant and a phenolic preservative above their concentration threshold, comprising including in the composition a solvent modifier in a concentration sufficient to ensure the composition remains clear.

The method of the above embodiment wherein the composition comprises any of the compositions described above.

Embodiments of the present invention are further described in the Examples below, which should not be construed as limiting.

### EXAMPLES

Concentration Threshold in Compositions Having 0.2 mg/mL PS80

The commercial formulations of dulaglutide marketed under the tradename TRULICITY® include 0.2 mg/mL of PS80 as stabilizer. In order to study the effects of the addition of a phenolic preservative, a placebo solution is prepared containing 0.2 mg/mL of PS80 in a 10 mM citrate buffer at pH 6.5, and test articles are prepared by adding sufficient quantities m-cresol or phenol to samples of this solution to result in formulations containing 0.2 mg/mL and either 3.15 mg/mL of m-cresol or 5 mg/mL of phenol. The placebo and test articles are inspected visually. Whereas the placebo solution is clear and colorless, the test articles each rapidly develop a cloudy or milky appearance. Thus, the concentration threshold was exceeded for each of the two preservative containing solutions.

Concentration Threshold in Compositions Containing m-Cresol and PS20

A study is conducted to determine the concentration threshold for combinations of PS20 and m-cresol, which are the non-ionic surfactant and phenolic preservative used in the commercial formulations of insulin glargine, marketed under the tradename LANTUS®, and insulin glulisine, marketed under the trade name APIDRA®, which include PS20 in concentrations of 0.02 mg/mL and 0.01 mg/mL and m-cresol in concentrations of 2.7 and 3.15 mg/mL, respectively. Placebo solutions are prepared in 10 mM phosphate buffer at pH 7 containing 3.15 mg/mL m-cresol and varying concentrations of PS20 ranging from ¼ up to 10× its CMC. The vials are analyzed by visual inspection. Results are provided in Table 1 below:

TABLE 1

| PS20 concentration (mg/mL) | Visual Appearance |
|---|---|
| ¼ × CMC (0.02 mg/mL) | Clear |
| ½ × CMC (0.04 mg/mL) | Clear |
| 1 × CMC (0.08 mg/mL) | Clear |
| 2 × CMC (0.16 mg/mL) | Clear |
| 5 × CMC (0.40 mg/mL) | Cloudy |
| 7 × CMC (0.56 mg/mL) | Cloudy |
| 10 × CMC (0.8 mg/mL) | Cloudy |

Results show that phase separation did not occur in these compositions when polysorbate 20 is included in concentrations at or below about 2 times its CMC, but does occur at concentrations at or above about 5 times its CMC. Thus, combinations of 3.15 mg/mL m-cresol and polysorbate 20 in concentration at or above 5 times its CMC are above the concentration threshold for m-cresol and polysorbate 20, whereas combinations of 3.15 mg/mL m-cresol and polysorbate 20 in concentrations at or below 2 times its CMC—e.g., the 0.02 and 0.01 mg/mL used in LANTUS and APIDRA—are below the concentration threshold for m-cresol and polysorbate 20.
Turbidity of Compositions Containing Varying Concentrations of m-Cresol and PS80

A study is performed to evaluate the relevance of concentration of both m-cresol and PS80 on the development of phase separation. A batch of 10-mM citrate buffer is prepared which contains, with pH adjusted to 6.5, and used as the control and buffer matrix for formulation of test articles. M-cresol is added to portions of the buffer matrix to prepare solutions having m-cresol in concentrations of 1.58 mg/mL, 2.70 mg/mL or 3.15 mg/mL. Polysorbate 80 is measured and dissolved in separate portions of the citrate buffer to prepare two stock solutions, one having 10 mg/mL polysorbate 80 and one having 40 mg/mL polysorbate. The stock solutions of surfactant are gradually added in the amounts indicated below in Table 2 to varying amounts of the phenolic preservative-containing solutions to generate formulations containing polysorbate 80 in a range of concentrations.

TABLE 2

| Polysorbate 80 (mg/mL) | Addition volume (μL) |
|---|---|
| 10 | 20 |
| | 50 |
| 40 | 50 |
| | 100 |

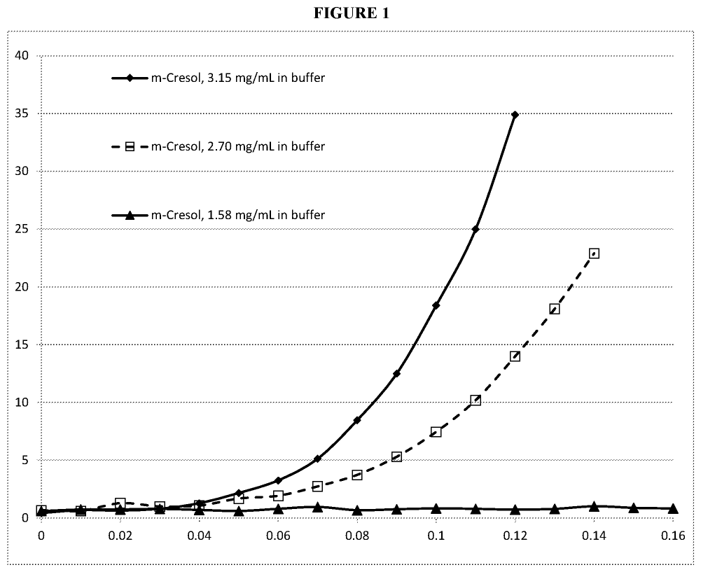

Turbidity of the resulting formulations is measured using a HACH turbidity meter (Model: 2100AN, Tag #: K349924). The instrument is calibrated using turbidity standards prior to use. A light coating of silicone oil is applied on the outer surface of the test tube to mask minor imperfections in glass tubes. Approximately 7 mL of solution is used for turbidity measurement. Results are provided in FIG. 1. As seen in FIG. 1, the development and magnitude of turbidity is dependent on the concentrations of both m-cresol and PS80.

### Effects of Varying Concentrations of Solvent Modifiers, Commonly Used Tonicity Agents, Preservatives and Surfactants.

Studies are performed to evaluate the impact of the inclusion of varying concentrations of solvent modifiers and other excipients, commonly used as tonicity agents in protein and peptide-based formulations, on preservative and surfactant compatibility in the solution state.

For one study, a batch of 10-mM phosphate buffer with pH adjusted to 6.5, and used as the buffer matrix. Subsequently, buffer solutions containing 3.15 mg/mL m-cresol and either a solvent modifier or a commonly used tonicity agent is prepared as set forth in Table 3.

TABLE 3

| | Concentration |
|---|---|
| Ingredient | (50 mg/mL) |
| Mannitol | 50.0 |
| Sodium chloride | 8.8 |
| PPG | 20.9 |
| Glycerol | 25.3 |
| NMP | 27.2 |
| PEG 400 | 110.0 |

Polysorbate 80 is measured and dissolved in the phosphate buffer to prepare two stock solutions, one having 10 mg/mL polysorbate 80 and one having 40 mg/mL polysorbate, which are gradually added in the amounts indicated above in Table 2 to varying amounts of the solvent modifier or tonicity agent-containing formulations described above in Table 3 to generate formulations of each containing polysorbate 80 in a range of concentrations. Turbidity of the resulting formulations is measured as described above.

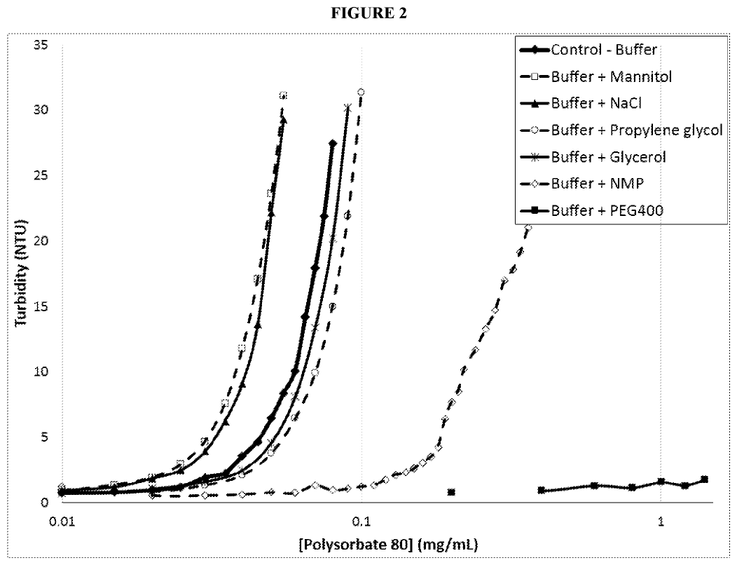

Results are provided in FIG. 2. As seen in FIG. 2, whereas the addition of mannitol and NaCl each result in a leftward shift of turbidity data as compared to control, indicating their inclusion lead to development of greater turbidity at given PS80 concentrations in this study, the addition of PPG, glycerol and NMP each resulted in a rightward shift of turbidity data as compared to control, and PEG 400 prevented the development of turbidity, indicating their inclusion attenuated the development of turbidity at given PS80 concentrations in this study.

For another set of studies, a 10-L batch of 10-mM citrate buffer is prepared which contains 2.723 mg/mL citric acid and 0.1422 sodium citrate, with pH adjusted to 6.5, and used as the buffer matrix. Subsequently, buffer solutions containing m-cresol and various excipients are prepared, as summarized in Table 4. Citric acid, sodium citrate dihydrate, polysorbate 80, m-cresol, liquefied phenol, mannitol and sodium chloride are obtained from Eli Lilly (Indianapolis, Ind.). Glycerol, propylene glycol, N-Methyl-2-pyrrolidone (NMP) and polyethylene glycol 400 (PEG 400) are obtained from Sigma-Aldrich (Milwaukee, Wis.).

TABLE 4
Composition of citrate buffer and placebo solutions

| | | Concentration | |
|---|---|---|---|
| ID | Ingredient | (mg/mL) | Description |
| 1 | m-Cresol | 3.15 | Control |
| 2 | m-Cresol | 3.15 | Mannitol - L |
| | Mannitol | 23 | |
| 3 | m-Cresol | 3.15 | Mannitol - M |
| | Mannitol | 46 | |
| 4 | m-Cresol | 3.15 | Mannitol - H |
| | Mannitol | 92 | |
| 5 | m-Cresol | 3.15 | NaCl - L |
| | Sodium chloride | 4.4 | |
| 6 | m-Cresol | 3.15 | NaCl - M |
| | Sodium chloride | 8.8 | |
| 7 | m-Cresol | 3.15 | NaCl - H |
| | Sodium chloride | 17.6 | |
| 8 | m-Cresol | 3.15 | Glycerol - L |
| | Glycerol | 20 | |
| 9 | m-Cresol | 3.15 | Glycerol - H |
| | Glycerol | 80 | |
| 10 | m-Cresol | 3.15 | Propylene glycol - L |
| | Propylene glycol | 15 | |
| 11 | m-Cresol | 3.15 | Propylene glycol - H |
| | Propylene glycol | 60 | |
| 12 | m-Cresol | 3.15 | NMP-L |
| | N-Methyl-2-pyrrolidone | 27 | |
| 13 | m-Cresol | 3.15 | NMP -M |
| | N-Methyl-2-pyrrolidone | 54 | |
| 14 | m-Cresol | 3.15 | NMP-H |
| | N-Methyl-2-pyrrolidone | 81 | |
| 15 | m-Cresol | 3.15 | PEG 400 - L |
| | Polyethylene glycol 400 | 40 | |
| 16 | m-Cresol | 3.15 | PEG 400 - M |
| | Polyethylene glycol 400 | 80 | |
| 17 | m-Cresol | 3.15 | PEG 400 - H |
| | Polyethylene glycol 400 | 120 | |
| 18 | m-Cresol | 1.58 | Mannitol - H |
| | Mannitol | 88 | |
| 19 | m-Cresol | 1.58 | NaCl - H |
| | Sodium chloride | 17.6 | |
| 20 | Phenol | 5 | Mannitol - M |
| | Mannitol | 46 | |
| 21 | Phenol | 5 | NaCl - M |
| | Sodium chloride | 8.8 | |

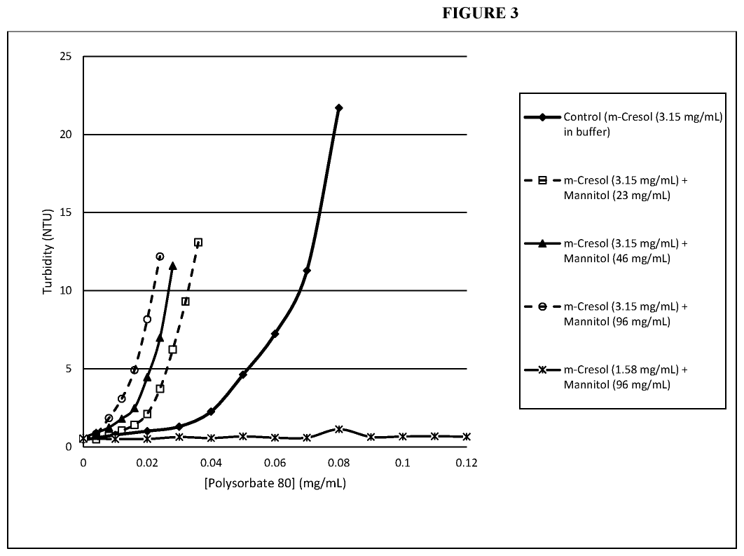

Polysorbate 80 is measured and dissolved in the phosphate buffer to prepare two stock solutions, one having 10 mg/mL polysorbate 80 and one having 40 mg/mL polysorbate, which are gradually added in the amounts indicated above in Table 2 to varying amounts of the solvent modifier or tonicity agent-containing formulations described above in Table 4 to generate formulations of each containing polysorbate 80 in a range of concentrations. Turbidity of the resulting formulations is measured as described above. Results are provided in FIGS. 3 through 8.

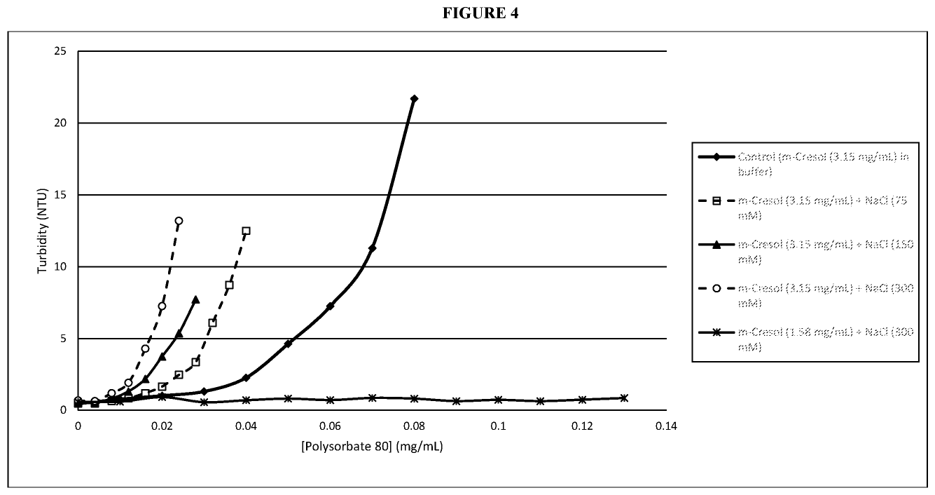

The contributions of both surfactant and preservative concentration and the deleterious effects of mannitol and NaCl can be seen in FIGS. 3 and 4. As seen in FIGS. 3 and 4, the formulation containing 1.58 mg/mL m-cresol is not turbid at any PS80 concentration tested, including in the presence of either mannitol or NaCl. Thus, the concentration threshold was not reached for any composition containing 1.58 mg/mL m-cresol tested in this study. When the concentration of m-cresol is increased to 3.15 mg/mL, however, the development of turbidity is seen as polysorbate 80 concentration is increased. Finally, the presence of either mannitol or NaCl exacerbates the development of turbidity in a dose-dependent manner.

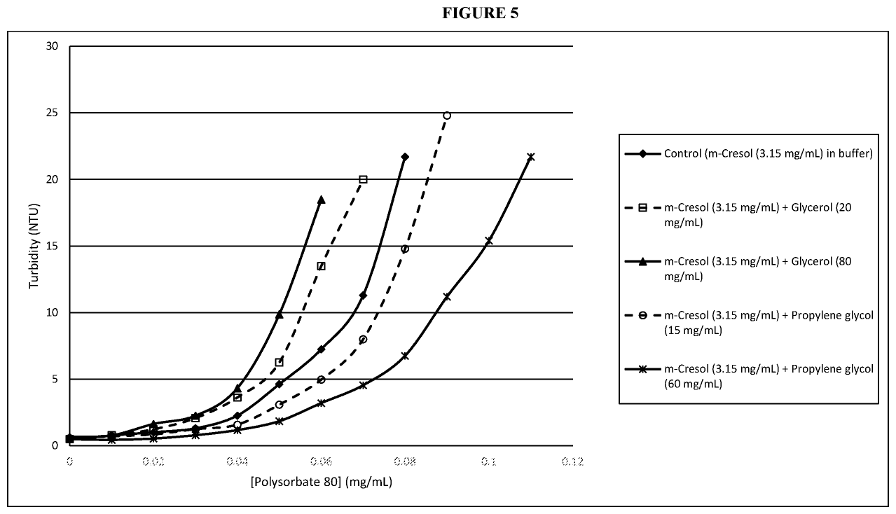

The impact of glycerol and PPG on the development of turbidity in certain surfactant and preservative concentrations can be seen in FIG. 5. As seen in FIG. 5, the inclusion of PPG attenuates the development of turbidity in a dose-dependent manner. Glycerol, on the other hand, resulted in a leftward shift in turbidity data as compared to control, suggesting it did not attenuate turbidity in the compositions tested in this study.

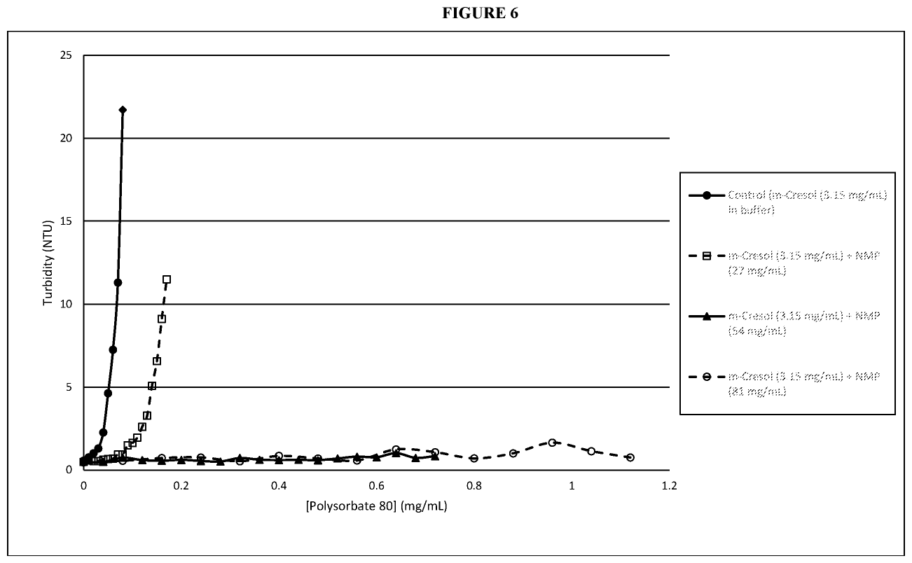

The impact of NMP can be seen in FIG. 6. As seen in FIG. 6, NMP attenuates the development of turbidity in a dose-dependent manner.

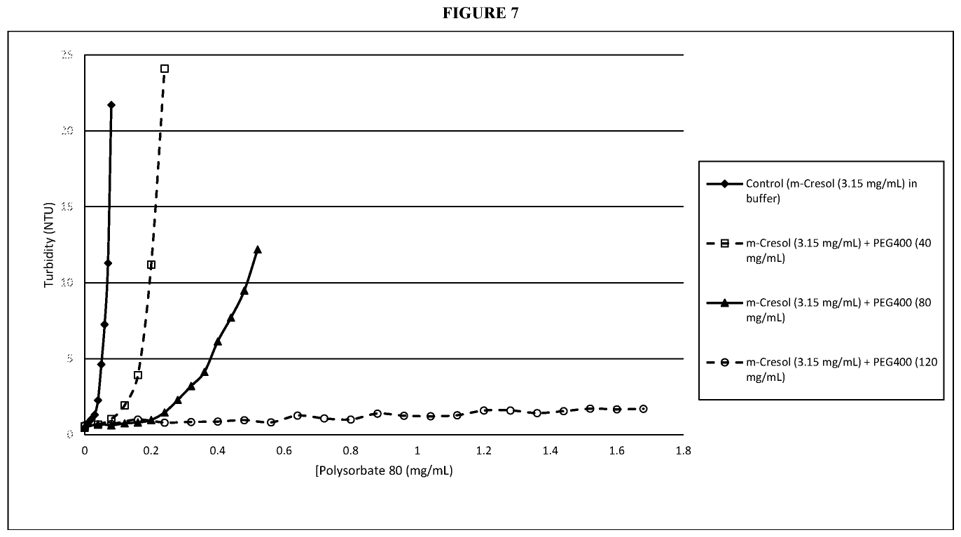

The impact of PEG400 on the point at which the concentration threshold is reached for certain PS80 and m-cresol concentrations can be seen in FIG. 7. As seen in FIG. 7, PEG400 attenuates the development of turbidity in a dose-dependent manner.

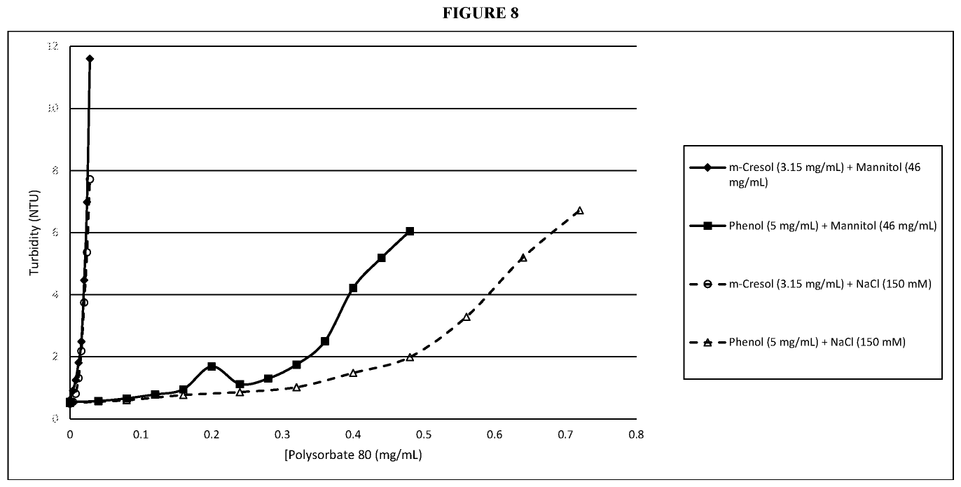

Finally, a comparison of the concentration thresholds for combinations of PS80 with m-cresol or phenol, in the presence of either mannitol or NaCl, can be seen in FIG. 8. As seen in FIG. 8, while both preservatives led to development of turbidity, phenol is more compatible with PS80 than m-cresol at all concentrations tested, and mannitol has a more deleterious effect than NaCl.

In sum, the data in these studies demonstrate that concentration thresholds are specific to the identities and concentrations of surfactants and preservatives in a composition, and that the development of phase separation resulting in turbidity in such compositions can be either attenuated in a dose-dependent manner through the inclusion of solvent modifiers or exacerbated in a dose-dependent manner through the inclusion of certain commonly-used isotonicity agents.

### Concentration Thresholds and Effects of Solvent Modifiers in Compositions Containing Model Proteins of Varying Molecular Weights

A study is conducted to confirm the interactions between surfactants and preservatives resulting in the development of turbidity in a composition, and the ability to attenuate that phenomenon through the inclusion of a solvent modifier, are not dependent on the identity of protein in the composition. The proteins identified for inclusion in this study are selected to include a range of molecular weights, as set forth in Table 5 below:

TABLE 5

| Protein | Molecular weight (kDa) |
|---|---|
| Thyroglobulin | 670 |
| Cytochrome c | 12.4 |
| C lysozyme | 14.3 |
| β-Lactoglobulin | 18.4 |
| Bovine serum albumin | 67 |

Sodium phosphate monobasic monohydrate, sodium phosphate dibasic heptahydrate, PS80, and m-cresol are obtained from Eli Lilly (Indianapolis, Ind.). N-Methyl-2-pyrrolidone (NMP), cytochrome C, lysozyme, β-lactoglobulin and thyroglobulin are obtained from Sigma-Aldrich (Milwaukee, Wis.). Bovine serum albumin is obtained from Akron. All ingredients are used as is.

A 2-L batch of 10-mM phosphate buffer is prepared by combining 0.7821 mg/mL sodium phosphate dibasic with 0.62 mg/mL sodium phosphate monobasic in water, with pH adjusted to 7.0, and used as the buffer matrix for the study. Subsequently, protein formulations containing PS80, m-cresol and/or NMP are prepared, and visually inspected. Details of the compositions and results are provided below in Table 6.

TABLE 6

| | | Polysorbate | m-Cresol | NMP | Visual |
|---|---|---|---|---|---|
| Protein (mg/mL) | | 80 (mg/mL) | (mg/mL) | (mg/mL) | Appearance |
| Cytochrome C, | | 0.2 | 0 | 0 | Clear |
| 5 | mg/mL | 0.2 | 3.15 | 0 | Cloudy |
| | | 0.2 | 3.15 | 81 | Clear |
| Lysozyme, | | 0.2 | 0 | 0 | Clear |
| 10 | mg/mL | 0.2 | 3.15 | 0 | Cloudy |
| | | 0.2 | 3.15 | 81 | Clear |
| β-Lactoglobulin, | | 0.2 | 0 | 0 | Clear |
| 10 | mg/mL | 0.2 | 3.15 | 0 | Cloudy |
| | | 0.2 | 3.15 | 81 | Clear |
| Bovine serum | | 0.2 | 0 | 0 | Clear |
| albumin, or | | 0.2 | 3.15 | 0 | Cloudy |
| 5 | mg/mL | 0.2 | 3.15 | 81 | Clear |
| Bovine serum | | 0.2 | 0 | 0 | Clear |
| albumin, | | 0.2 | 3.15 | 0 | Cloudy |
| 10 | mg/mL | 0.2 | 3.15 | 81 | Clear |
| Thyroglobulin | | 0.2 | 0 | 0 | Clear |
| 5 | mg/mL | 0.2 | 3.15 | 0 | Cloudy |
| | | 0.2 | 3.15 | 81 | Clear |

The data in Table 6 demonstrate that for all proteins tested, including multiple concentrations of BSA, the combination of 0.2 mg/mL polysorbate 80 and 3.15 mg/mL m-cresol in the absence of any solvent modifier causes phase separation resulting in a cloudy appearance, while the inclusion of 81 mg/mL NMP prevents the development of such phase separation.

### Stability Study on Preserved Formulations of Dulaglutide

A study is designed to test the stability of preserved formulations of dulaglutide prepared with solvent modifiers according to the present invention. The currently available commercial formulation of TRULICITY® (dulaglutide) contains 3 mg/mL dulaglutide, 0.2 mg/mL PS80 and 46.4 mg/mL mannitol in a 10 mM citrate buffer, pH 6.5. As noted above, previous efforts to preserve this formulation through the addition of a phenolic preservative resulted in phase separation due to incompatibility between the PS80 and the phenolic preservative. Through the use of solvent modifiers as described herein, however, modified formulations were developed which allow for the inclusion of preservative(s) sufficient to achieve sufficient antimicrobial effectiveness, and the 0.2 mg/mL PS80 necessary for stability purposes, but without the phase separation observed for non-solvent modifier-containing formulations. The compositions of those formulations are set forth below in Table 7.

TABLE 7

| | | | | Tonicity | |
|---|---|---|---|---|---|
| ID | Buffer | Dulaglutide | PS 80 | agent | Preservative |
| A | 10 mM | 3 mg/mL | 0.2 | Propylene | Phenol, 4 mg/mL |
| | citrate | | mg/mL | glycol, | |
| | buffer, | | | 15 mg/mL | |
| B | pH = 6.5 | | | Propylene | m-Cresol, 0.32 |
| | | | | glycol, | mg/mL & phenol, |
| | | | | 15 mg/mL | 3.5 mg/mL |
| C | | | | Glycerol, | Phenol, 4 mg/mL |
| | | | | 20 mg/mL | |
| D | | | | Glycerol, | m-Cresol, 0.32 |
| | | | | 20 mg/mL | mg/mL & phenol, |
| | | | | | 3.5 mg/mL |

A study is designed to test the stability of dulaglutide in these compositions. Citrate buffer at 5 mM, pH=6.5 is prepared and used as is. An appropriate amount of citrate buffer is transferred to a 500-mL volumetric flask. Calculated amounts of preservative and solvent modifier are then added to the same flask, and mixed to dissolve to ensure a homogeneous solution. Using a graduated cylinder, 38.5 mL of dulaglutide drug substance is measured, and transferred to the volumetric flask. The solution is mixed until homogeneous. Concurrently, a stock solution of polysorbate 80 at 100 mg/mL is prepared. Approximately 1000 mg of polysorbate is transferred into a glass beaker, and dissolved in 10 mL of buffer solution. Using a transfer pipet, 1 mL of the polysorbate 80 stock solution is transferred to the volumetric flask. Appropriate amount of buffer is then added until the liquid meniscus reaches the 500-mL mark. Solution is further mixed to ensure homogeneity, and filtered through a 0.22-μm filter. The filtered drug product is filled into 3-mL cartridges. Solution in the cartridges is visually confirmed to be clear, indicating phase separation due to surfactant and preservative interaction has not occurred.

In addition, filled cartridges are stored at 5° C. for stability testing. The 5° C. storage temperature is representative of the recommended storage temperature of 2-8° C. for dulaglutide drug product. At pre-designated timepoints, samples are removed from storage, confirmed visually to be clear and particulate free, and tested with various methods as described below.

HIAC. HIAC testing is used to measure subvisible particulate content, and is performed on test samples as described in USP <787>(Subvisible Particulate Matter in Therapeutic Protein Injections) and <788>(Particulate Matter in Injections), which are harmonized with European Pharmacopeia 2.9.19 and Japanese Pharmacopeia 6.07. For each time point, 5 aliquots of 0.5 mL of solution are withdrawn from a 3 mL cartridge and pooled, so the measured result(s) reflect an average of 5 samples. Results are provided below in Table 8.

TABLE 8
HIAC results.

| | | Particulate (part/mL) | | | | | | | |
|---|---|---|---|---|---|---|---|---|---|
| | Time | 2 μm | | 5 μm | | 10 μm | | 25 μm | |
| | point | | Std. | | Std. | | Std. | | Std. |
| Sample | (month) | Value | Dev. | Value | Dev. | Value | Dev. | Value | Dev. |
| A | 0 | 2960 | 87 | 1620 | 76 | 602 | 25 | 14 | 5 |
| | 0.5 | 4135 | 92 | 1941 | 110 | 702 | 18 | 15 | 5 |
| | 1 | 818 | 76 | 314 | 33 | 105 | 18 | 3 | 3 |
| | 2 | 2717 | 60 | 1146 | 39 | 366 | 6 | 22 | 6 |
| | 3 | 2560 | 61 | 761 | 56 | 139 | 13 | 11 | 3 |
| | 6 | 2720 | 79 | 1132 | 51 | 406 | 48 | 18 | 6 |
| | 12 | 3830 | 56 | 1688 | 32 | 415 | 25 | 7 | 5 |
| B | 0 | 6910 | 30 | 3239 | 90 | 1149 | 76 | 14 | 5 |
| | 0.5 | 2288 | 109 | 740 | 48 | 204 | 18 | 5 | 3 |
| | 1 | 6753 | 124 | 3456 | 94 | 1200 | 16 | 14 | 3 |
| | 2 | 3402 | 49 | 1442 | 26 | 356 | 27 | 18 | 8 |
| | 3 | 4487 | 26 | 1640 | 55 | 433 | 15 | 13 | 2 |
| | 6 | 1847 | 4 | 518 | 54 | 98 | 0 | 9 | 4 |
| | 12 | 7182 | 331 | 2883 | 347 | 552 | 183 | 18 | 16 |
| C | 0 | 1648 | 53 | 781 | 27 | 312 | 29 | 25 | 7 |
| | 0.5 | 6269 | 169 | 2671 | 116 | 423 | 37 | 2 | 2 |
| | 1 | 4927 | 29 | 1574 | 49 | 225 | 66 | 1 | 2 |
| | 2 | 6215 | 40 | 2600 | 21 | 831 | 35 | 40 | 9 |
| | 3 | 3870 | 83 | 1710 | 44 | 332 | 10 | 4 | 2 |
| | 6 | 5619 | 69 | 2347 | 10 | 517 | 4 | 3 | 4 |
| | 12 | 6586 | 363 | 2944 | 357 | 741 | 116 | 20 | 8 |
| | 1 | 5465 | 641 | 2331 | 123 | 773 | 44 | 23 | 10 |
| D | 0 | 2421 | 167 | 1076 | 61 | 334 | 20 | 7 | 4 |
| | 0.5 | 14595 | 46 | 4539 | 33 | 842 | 98 | 13 | 6 |
| | 1 | 7640 | 191 | 3490 | 97 | 710 | 36 | 1 | 1 |
| | 2 | 1624 | 16 | 555 | 32 | 185 | 14 | 9 | 7 |
| | 3 | 6388 | 96 | 3284 | 121 | 1078 | 68 | 22 | 4 |
| | 6 | 1259 | 18 | 399 | 1 | 113 | 10 | 10 | 0 |
| | 12 | 3636 | 210 | 1467 | 197 | 397 | 90 | 31 | 7 |

Compliance with USP <788>(Particulate Matter in Injections) requires parenteral products containing therapeutic protein injections, such as dulaglutide, to have no greater than 6000 particulates equal to or greater than 10 μm and no greater than 600 particulates equal to or greater than 25 μm per container. As seen in Table 8, all samples tested were well-within FDA limits for parenteral products.

MFI. MFI testing is used to detect particulate matter present in injections and parenteral solutions, other than gas bubbles. This method is a stability indicating characterization method for information only, and is performed for the purpose of enumerating and categorizing sub-visible particles with respect to size, concentration, and morphology using flow imaging technology. Samples are withdrawn from storage and tested after 12 months. Results are provided in Table 9 below. Particulates greater than or equal to 5 μm with an aspect ratio (AR) greater than 0.85 are highly circular in shape, and are likely to be silicone from the stopper as opposed to particles of protein.

TABLE 9

| | Particulate (part/mL) | | | |
|---|---|---|---|---|
| ID | ≥2 μm | ≥5 μm | ≥5 μm & > 0.85 AR | CF |
| A | 11592 | 2590 | 1937 | 0.75 |
| B | 10158 | 1839 | 1427 | 0.78 |
| C | 8525 | 1667 | 1517 | 0.91 |
| D | 7028 | 1233 | 925 | 0.75 |

Abbreviations: AR—aspect ratio; CF—circular fraction.

Data in Table 9 are comparable to those for historical dulaglutide drug product.

SEC. A size-exclusion (SEC) HPLC method is used to measure the monomer purity of dulaglutide. This method separates aggregates and fragmented species from the intact, monomeric protein.

Monomer purity in dulaglutide drug product is determined by Size Exclusion HPLC. The method uses isocratic separation on a 200 angstrom pore size silica gel column with UV detection at 214 nm, which is near the absorbance maximum of the peptide backbone of the drug product and thus no correction for response factors is necessary. High molecular weight forms (Total aggregates) are separated from monomeric dulaglutide by this method. The method has been demonstrated to be specific and stability indicating; it separates high molecular weight forms from the dulaglutide monomer. Monomer and aggregates are reported as peak area percent to the total area. Data are provided in Table 10.

TABLE 10

| | Time (month) | | | | | | | Time (month) | | | | | | |
|---|---|---|---|---|---|---|---|---|---|---|---|---|---|---|
| | Monomer (%) | | | | | | | Total aggregates (%) | | | | | | |
| ID | 0 | 0.5 | 1 | 2 | 3 | 6 | 12 | 0 | 0.5 | 1 | 2 | 3 | 6 | 12 |
| A | 99.2 | 99.0 | 98.3 | 98.6 | 98.1 | 98.2 | 98.6 | 0.8 | 1.0 | 1.2 | 1.2 | 1.3 | 1.6 | 1.4 |
| B | 98.7 | 98.7 | 98.4 | 98.5 | 98.2 | 98.0 | 98.2 | 1.3 | 1.3 | 1.6 | 1.5 | 1.8 | 2.0 | 1.8 |
| C | 99.1 | 99.0 | 98.3 | 98.6 | 97.8 | 98.0 | 98.1 | 0.9 | 1.0 | 1.2 | 1.2 | 1.5 | 1.8 | 1.9 |
| D | 99.0 | 98.9 | 98.3 | 98.5 | 97.8 | 97.9 | 97.9 | 1.0 | 1.1 | 1.3 | 1.3 | 1.5 | 1.9 | 2.1 |

The data in Table 10 are within acceptance limits for dulaglutide drug product.

RP-HPLC. This method is designed for the determination of purity and related substances/impurities in dulaglutide drug product. Related impurities resulting from aglycosylation, N-terminal truncation, linker clipping and Fc region oxidation are separated from unmodified dulaglutide using reversed-phase gradient HPLC with UV detection at 214 nm, which is near the absorbance maximum of the peptide backbone of the drug product, and thus no correction for response factors is necessary. The method has been demonstrated to be specific and stability indicating; it separates degradation products from the main peak.

TABLE 11
RP-HPLC results.

| | Main peak (%) | | | | | | | Linker clipping (%) | | | | | | |
|---|---|---|---|---|---|---|---|---|---|---|---|---|---|---|
| | Time (month) | | | | | | | Time (month) | | | | | | |
| ID | 0 | 0.5 | 1 | 2 | 3 | 6 | 12 | 0 | 0.5 | 1 | 2 | 3 | 6 | 12 |
| A | 89.3 | 88.8 | 89.2 | 89.7 | 87.8 | 87.6 | 85.3 | 0.5 | 0.6 | 0.6 | —* | 0.8 | 1.0 | 1.5 |
| B | 90.2 | 89.5 | 89.8 | 88.5 | 88.5 | 88.5 | 86.9 | 0.1 | —* | 0.1 | 0.7 | 0.1 | 0.1 | 0.2 |
| C | 89.4 | 88.9 | 89.0 | 89.4 | 87.6 | 87.2 | 85.0 | 0.5 | 0.6 | 0.6 | —* | 0.8 | 1.0 | 1.5 |
| D | 89.1 | 88.9 | 89.0 | 88.1 | 87.5 | 87.1 | 84.4 | 0.5 | 0.6 | 0.6 | 0.7 | 0.8 | 1.0 | 1.5 |

*Data not available due to analytical error.

The data in Table 11 are within acceptance limits for dulaglutide.

Limited digest. A limited digest method is designed for the determination of modifications to the GLP-1 analog in dulaglutide drug product. The drug product sample is exposed to mild digestion conditions with trypsin to free the GLP-1 analog and linker from the Fc portion of the molecule. The GLP-1 analog is digested into three smaller peptides. The method uses reversed-phase gradient HPLC separation with UV detection at 214 nm, which is near the absorbance maximum of the peptide backbone of the drug product and thus no correction for response factors is necessary. Related impurities resulting from N-terminal truncation, N-terminal modifications (Des H/HG, pyruvylation), oxidation of tryptophan at position 25 and hydroxylation of lysine at position 28 are separated from unmodified dulaglutide peptides by this method. The method has been demonstrated to be specific and stability indicating; it separates related substances and impurities from the respective unmodified peptides. Results are provided in Table 12.

TABLE 12
Limited digest results.

| | des H/HG (%) | | | | | | | Total Hydrox/Oxidized (%) | | | | | | |
|---|---|---|---|---|---|---|---|---|---|---|---|---|---|---|
| | Time (month) | | | | | | | Time (month) | | | | | | |
| ID | 0 | 0.5 | 1 | 2 | 3 | 6 | 12 | 0 | 0.5 | 1 | 2 | 3 | 6 | 12 |
| A | 2.7 | 2.3 | 2.7 | 2.7 | 2.5 | 2.4 | 2.7 | 4.4 | 4.1 | 3.9 | 4.1 | 4.1 | 4.2 | 4.3 |
| B | 2.7 | 2.4 | 2.7 | 2.6 | 2.4 | 2.4 | 2.6 | 4.3 | 4.2 | 3.8 | 4.1 | 4.0 | 4.2 | 4.4 |
| C | 2.8 | 2.4 | 2.7 | 2.7 | 2.5 | 2.5 | 2.4 | 4.3 | 4.1 | 3.9 | 4.0 | 4.0 | 4.2 | 4.5 |
| D | 2.7 | 2.4 | 2.7 | 2.7 | 2.5 | 2.5 | 2.6 | 4.3 | 4.1 | 3.8 | 4.0 | 4.0 | 4.2 | 4.3 |

Abbreviations - des H/HG: N-terminal truncation, N-terminal modifications; Hydrox/Oxidized: oxidation of tryptophan at position 25 and hydroxylation of lysine at position 28.

The data in Table 11 are within acceptance limits for dulaglutide.

CE-SDS NR. Capillary electrophoresis sodium dodecyl sulfate non-reduced (CE-SDS NR) testing is used to determine purity in dulaglutide drug product. The dulaglutide molecule is denatured and the molecular variants are separated by size via a proprietary gel matrix that is electrokinetically loaded into an uncoated capillary. Separation occurs when an electric current is applied to the capillary and molecular variants are detected by UV at 214 nm, which is near the absorbance maximum of the peptide backbone of the drug product and thus no correction for response factors is necessary. High molecular weight and single chain forms are separated from monomeric dulaglutide by this method. The method has been demonstrated to be specific and stability indicating; it separates aggregate and single chain from the dulaglutide monomer.

TABLE 13

| | GLP-Fc main peak (%) | | | | | | |
|---|---|---|---|---|---|---|---|
| | Time (month) | | | | | | |
| ID | 0 | 0.5 | 1 | 2 | 3 | 6 | 12 |
| A | 97.1 | 97.2 | 96.7 | 96.6 | 96.6 | 96.5 | 95.8 |
| B | 96.7 | 96.8 | 96.3 | 96.5 | 96.2 | 96.1 | 95.5 |
| C | 97.1 | 97.0 | 96.7 | 96.7 | 96.6 | 96.2 | 95.6 |
| D | 97.1 | 97.0 | 96.6 | 96.6 | 96.6 | 96.2 | 95.2 |

The data in Table 13 are within acceptance limits for dulaglutide drug product.

In sum, the above studies support the conclusion that preserved formulations of dulaglutide, which contain the same PS80 content used to provide sufficient stability in the currently available commercial formulation of TRULICITY may be prepared without phase separation due to preservative-surfactant interactions through the use of solvent modifiers, and that the protein in such formulations remains sufficiently stable.

1.-26. (canceled)

27. A composition comprising:
a) dulaglutide;
b) polysorbate 80 (PS80);
c) a phenolic preservative selected from the group consisting of phenol, m-cresol and mixtures thereof; and
d) a solvent modifier selected from the group consisting of N-methyl-2-pyrrolidone (NMP), polyethylene glycol (PEG) 400, propylene glycol (PPG) and glycerol.

28. The composition of claim 27 wherein the concentration of PS80 is about 0.2 mg/mL.

29. The composition of claim 28 wherein the dulaglutide concentration is selected from the group consisting of about 1.5 mg/mL, about 3 mg/mL, about 6 mg/mL and about 9 mg/mL.

30. The composition of claim 29 wherein the phenolic preservative is about 4 mg/mL phenol.

31. The composition of claim 30 wherein the phenolic preservative is a combination of about 3.5 mg/mL phenol and about 0.32 mg/mL m-cresol.

32. The composition of claim 31, wherein the solvent modifier is PPG.

33. The composition of claim 32 wherein the concentration of PPG is about 15 mg/mL.

34. The composition of claim 31, wherein the solvent modifier is glycerol.

35. The composition of claim 34 wherein the concentration of glycerol is about 20 mg/mL.

36. The composition of claim 33 further comprising a 10 mM citrate buffer and wherein the pH of the composition is 6.5.

37.-39. (canceled)

41. The composition of claim 35 further comprising a 10 mM citrate buffer and wherein the pH of the composition is 6.5.
